(19) United States
(12) Patent Application Publication
(10) Pub. No.: US 2023/0067787 A1
(43) Pub. Date: Mar. 2, 2023

(54) HYDRO-CONTACT LENS

(71) Applicant: Miguelangel Salazar, Aventura, FL (US)

(72) Inventor: Miguelangel Salazar, Aventura, FL (US)

(21) Appl. No.: 17/823,351

(22) Filed: Aug. 30, 2022

Related U.S. Application Data
(60) Provisional application No. 63/238,575, filed on Aug. 30, 2021.

(51) Int. Cl. A61F 9/00; A61F 9/02

(52) U.S. Cl. CPC A61F 9/0017; A61F 9/029; A61F 2009/021

(57) ABSTRACT

An eye treatment device includes the following: a goggle frame, having at least one cavity, configured to surround and be spaced from an eye; a corrective lens along a face of the goggle frame; and an inlet formed in an upper portion of the goggle frame, the inlet fluidly communicating the cavity to an external environment; further including: a sealing material along a perimeter of the goggle frame for forming a hermetical seal against skin around a socket of the eye, wherein the sealing material is a portion of a suction cup; and further including a tapered stopper for fluidly sealing the inlet, wherein the corrective lens is made from gas permeable material.

### CROSS-REFERENCE TO RELATED APPLICATION

This application claims the benefit of priority of U.S. provisional application No. 63/238,575, filed 30, Aug. 2021, the contents of which are herein incorporated by reference.

### BACKGROUND OF THE INVENTION

The present invention relates to ophthalmic lenses and, more particularly, an optical device for hydration of the eyeball, wherein the optical device includes a gas permeable corrective screen fixed to a frame, wherein the corrective screen is dimensioned and adapted to adhere to the eye socket to form a liquid chamber so that, when filled with isotonic solution, becomes a liquid body contacting the entirety of the exposed eye and thereby correcting atypical ametropia, which is impossible for conventional ophthalmic lenses.

The most frequent ametropias include myopia, hyperopia, astigmatism, presbyopia, irregular astigmatism, irregularity after refractive surgery or corneal implants with remaining distortion of corneal curvature, keratoconus, marginal degeneration, and corneal trauma that leave scars. Other important conditions include dry eye syndrome, low vision, strabismus, phorias, and marked anisometropia.

The only existing device that claims to address the problem of dry eye resulting from the lack of proper hydration of the eyeball is the corneal scleral device (scleral support contact lens), putatively providing corrective vision. However, it is not entirely true, since the hydration it generates is limited to the diameter of the corrective lens that directly engages the eye, the standard scleral support lens having a diameter of approximately 25 millimeters. Thus, it is not correct to say that the corneal scleral support device dominates the dry eye syndrome. On the other hand, the correction of atypical ametropia, until now, has been the exclusive use of rigid and specialized contact lenses.

Existing devices or systems (specifically, the use of contact lenses) do not work 100% in the case of dry eye syndrome. Specifically, the scleral support contact lens only provides hydration within its circumferential bounds, and thus the portion of the wearer's eye outside that circumference is untreated. Furthermore, contact lenses, of any type, cannot be used in cases of infections or ocular affections, as well as in cases where the patient must undergo specialized studies. Similarly, contact lenses do not work well in cases where the patient needs to rest his eyes, after a reasonable time of use. Likewise, contact lenses do not work well in cases where the patient, due to a disability or simply because he/she does not have the required dexterity due to some factor that prevents it (e.g., nerves, restlessness), is not capable of inserting the contact lenses by him/herself.

As can be seen, there is a need for an optical device for hydration of the entirety of the exposed eyeball, wherein the optical device includes a gas permeable corrective screen fixed to a frame, wherein the corrective screen is dimensioned and adapted to adhere to the eye socket so as to define a substantially hermetically sealed liquid chamber therearound that, when filled with isotonic solution, becomes a liquid volume that envelopes the entirety of the exposed eye, correcting dehydration-related ailments of the eye along an entirety of the exposure portions of the eye. The hydro-contact lens apparatus embodied in the present invention can correct refractive ametropia, as well as conventional and atypical vision, through completely hydration of the anterior segment of the eyeball up to its attachments, without any diameter limitation. It can also be used for ophthalmic medication treatments that require exposure for specific times without loss of dose. All these problems are addressed with high performance. Likewise, when there are infections or bacterial eye conditions in the eye, the first thing that is recommended to the patient is that he/she cannot use contact lenses during the ophthalmic treatment. The hydro-contact spectacle solves this problem, since the patient can use it during the treatment and maintain the vision, since the hydro-contact spectacle/goggle is not in contact with the anterior segment of the eyeball and so it can be kept in use during the corresponding medical treatment and provide the necessary vision to the patient. Finally, in the case of patients with atypical ametropia who must undergo specialized studies, for example, corneal topography, where they are prevented from using contact lenses and are considered disabled, the hydro-contact spectacle is the solution to the problem, since contact lenses cannot be used either before or during these studies.

### SUMMARY OF THE INVENTION

The hydro-contact optical device embodied in the present invention is a visual correction device, which differs in its structure, mainly because it has no physical contact with the cornea, and only water contact on the surface of the anterior segment of the eyeball, which makes the invention unique, novel and different from everything that exists and, together with the other existing devices, the hydro-contact eyeglass is the best alternative for patients who require it, especially for the most visually impaired.

In the case of atypical ametropias, the present invention becomes a lens that corrects and resolves irregular refractions, thus generating a coherent refraction, being molded against the inner face of the corrective shield and being contained in front of the cornea, it becomes a second lens, and this liquid containment neutralizes the irregular refraction, allowing the optical correction of all the aforementioned visual defects to be resolved.

Likewise, in these cases, the hydro-contact spectacle component, called the corrective shield, has the capacity to generate the necessary diopters, this is achieved by its internal base curvature and by its corrective carving on the posterior face.

As for other important conditions, the hydro-contact spectacle lens solves them as follows: (i) the liquid interaction, provides permanent hydration to the eyeball for the case of dry eye syndrome, of any scale, (ii) the focal distance in front of the cornea can be varied in case certain magnifications condition, such as in cases of low vision, and (iii) as for the dioptric power correction, it is handled by making variations of the base curve of the corrective shield and, additionally, with the dioptric power that is calculated to the corrective shield.

In the case of treatments with ophthalmic medication, the present invention enables medication to be diluted in the liquid chamber so that it has an interaction with the anterior segment of the eyeball, and since the prescribed medication is contained in the fluidly sealed liquid chamber, there is no possibility of drainage, thus the patient could achieve an optimal recovery.

The hydro-contact goggle solves and selectively attends the optical dysfunctions or syndromes, such as dry eye syndrome. In cases where treatment or study of the eye is required, the present invention allows patients to have a device within their reach that provides vision during the time of the treatment and/or study. The present invention is an optical device that works correctly. In short, the hydro-contact spectacle solves the cases where the patient, for the reasons described above, cannot wear contact lenses.

The hydro-contact spectacle solves the dry eye syndrome problem through completely hydrating the entirety of anterior segment of the eyeball up to its attachments, without any diameter limitation.

Now, with the present invention of the hydro-contact spectacle, not only is a new field being born, but it also has the ability to resolve ametropia without the sensation of a foreign body. Likewise, when there are infections or bacterial conditions in the eye, the first thing that is recommended to the patient is that he/she cannot use contact lenses during the ophthalmic treatment. The hydro-contact spectacle obviates this restriction, as the patient can use it during the treatment and also maintain the vision, since the hydro-contact spectacle is not in contact with the anterior segment of the eyeball.

In one aspect of the present invention, a goggle or goggles can be configured to fit over one or both eyes of a patient and seal against the skin, around the eye sockets. The goggle or goggles can be sufficiently airtight to allow a desired air pressure to be maintained in one or more cavities or liquid chambers inside the goggle or goggles and over the eye(s) and hermetically sealed from the external environment by said airtight connection around the eye socket(s). There can be a seal material 121 positioned around a perimeter of the goggle frame, which can create a seal between edges of the goggle or goggles and the patient's skin around the eyes.

In another aspect of the present invention, an eye treatment device includes the following: a goggle frame, having at least one cavity, configured to surround and be spaced from an eye; a corrective lens along a face of the goggle frame; and an inlet formed in an upper portion of the goggle frame, the inlet fluidly communicating the cavity to an external environment; further including: a sealing material along a perimeter of the goggle frame for forming a hermetical seal against skin around a socket of the eye, wherein the sealing material is a portion of a suction cup; and further including a tapered stopper for fluidly sealing the inlet, wherein the corrective lens is made from gas permeable material having a thickness of approximately 0.70 millimeters, wherein a distance between a distance between a back surface of the corrective lens and a front of a of the eye is selected as function of a radius of curvature of the corrective lens.

In yet another aspect of the present invention a method of treating dry eye syndrome, the method includes providing the above-mentioned eye treatment device and filling the cavity with a fluid by way of the inlet.

These and other features, aspects and advantages of the present invention will become better understood with reference to the following drawings, description, and claims.

### BRIEF DESCRIPTION OF THE DRAWINGS

FIG. 1 is a top rear perspective view of an exemplary embodiment of the present invention.

FIG. 2 is an exploded top front perspective view of an exemplary embodiment of the present invention.

FIG. 3 is an elevation view of an exemplary embodiment of a strap 7 of the present invention.

FIG. 4 is an exploded bottom perspective view of an exemplary embodiment of a bridge 6 of the present invention.

FIG. 5 is an exploded bottom perspective view of an exemplary embodiment of a corrective screen 1 of the present invention. Illustrating the he radius of curvature R, the diameter A, and the height A of the corrective screen 1.

FIG. 6 is a lateral view of an exemplary embodiment of a frame 2 of the present invention.

FIG. 7 is a superior view of an exemplary embodiment of the frame 2 of the present invention.

FIG. 8 is a section view of an exemplary embodiment of the frame 2 of the present invention, taken along line 8-8 in FIG. 7.

FIG. 9 is an elevation view of an exemplary embodiment of a rubber gasket 8 of the present invention.

FIG. 10 is a section view of an exemplary embodiment of the frame 2 of the present invention, taken along line 10-10 in FIG. 11.

FIG. 11 is a superior view of an exemplary embodiment of the suction cup 5 of the present invention.

FIG. 12 is a section view of an exemplary embodiment of the present invention showing the formation of the liquid chamber/cavity around the eye 11 of the user.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, the present invention includes an eye treatment device includes the following: a goggle frame 2, having at least one cavity, configured to surround and be spaced from an eye; a corrective lens along a face 22 of the goggle frame; and an inlet formed in an upper portion of the goggle frame, the inlet fluidly communicating the cavity to an external environment; further including: a sealing material 54 along a perimeter of the goggle frame for forming a hermetical seal against skin around a socket of the eye, wherein the sealing material is a portion of a suction cup; and further including a tapered stopper for fluidly sealing the inlet, wherein the corrective lens is made from gas permeable material having a thickness of approximately 0.70 millimeters, wherein a distance between a distance between a back surface of the corrective lens and a front of a of the eye is selected as function of a radius of curvature of the corrective lens.

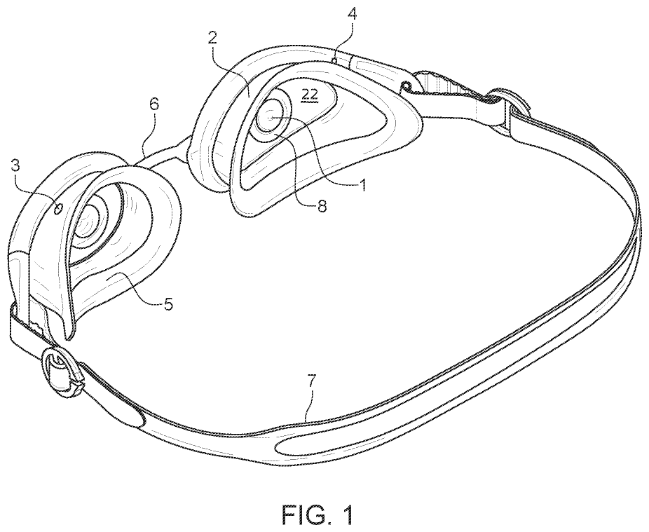

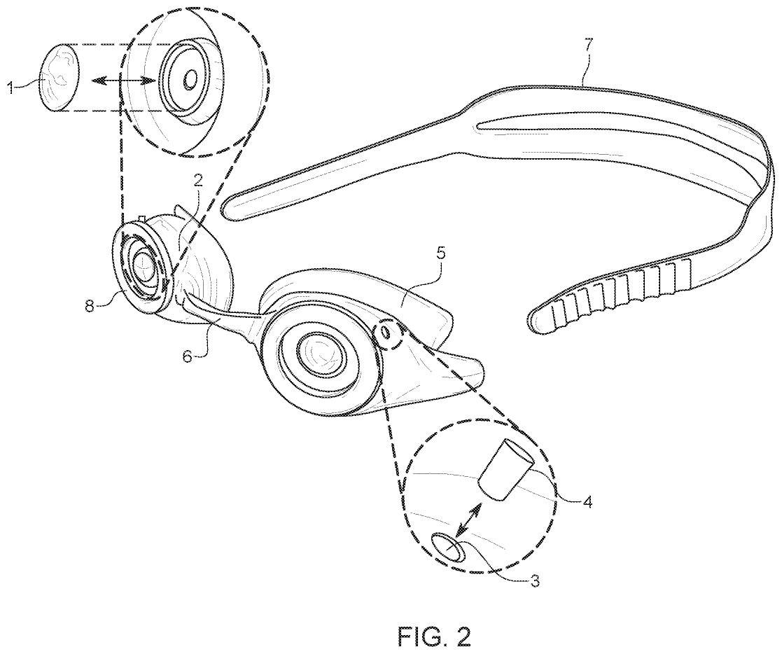

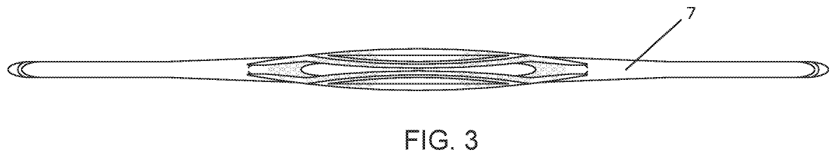

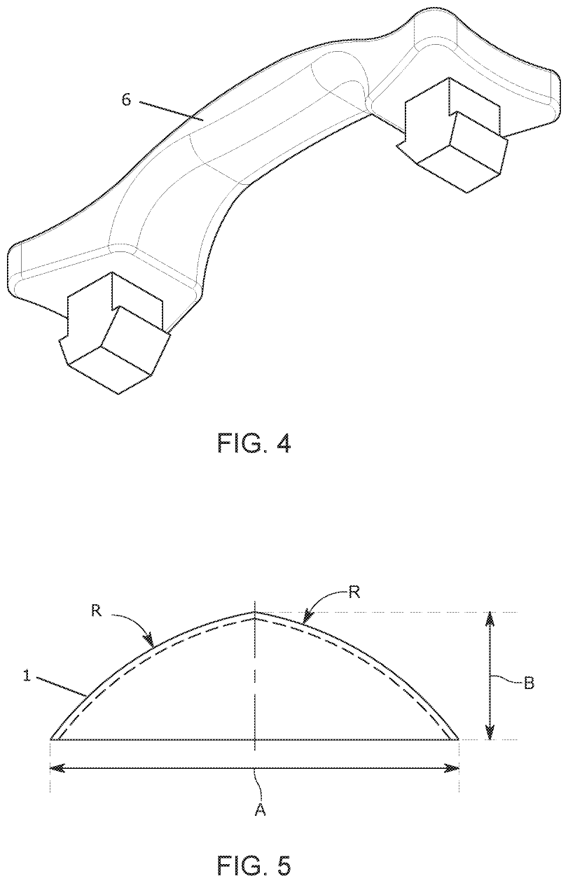

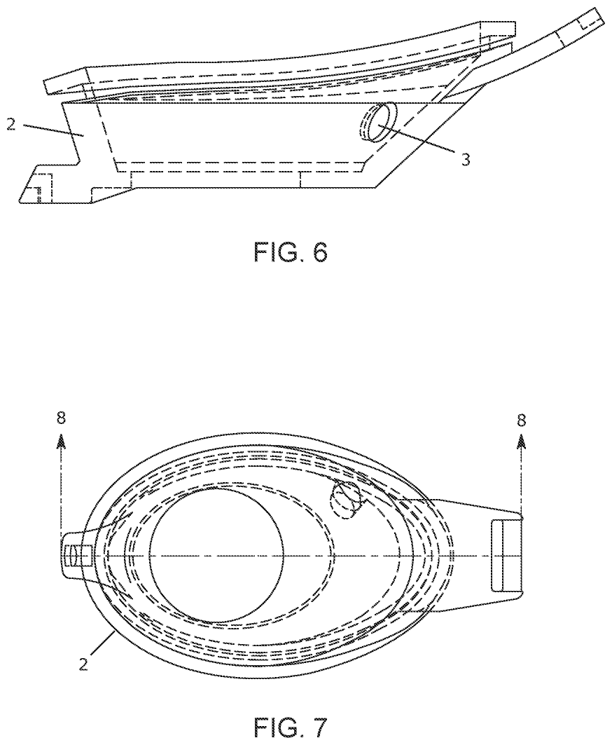

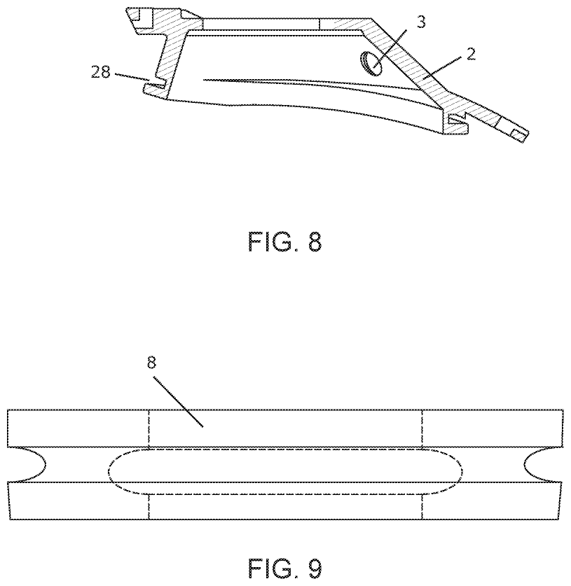

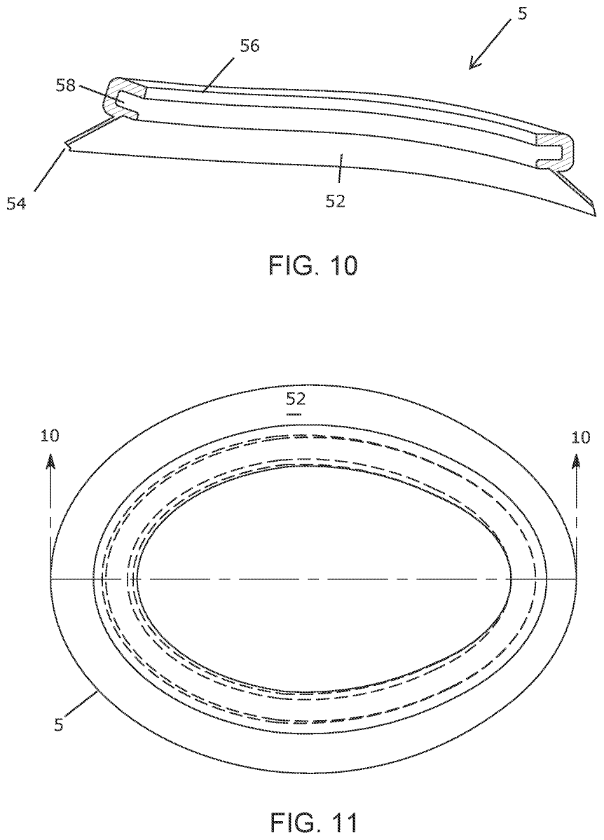

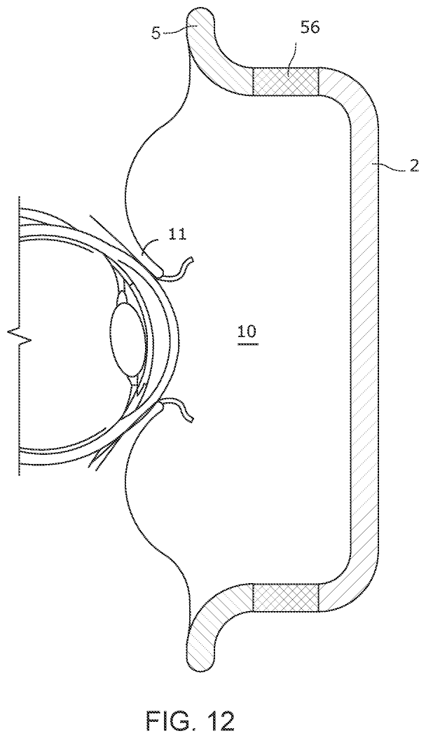

Referring now to FIGS. 1 and 12, the hydro-contact lens assembly 100 of the present invention may include one or two gas permeable corrective screens 1, each corrective screen 1 dimensioned and adapted to correct diopters. Each gas permeable corrective screen 1 has a form and shape determined by the carving of the material on its external face, as well as by the water exchange of its internal base curvature. This material may vary in diameter and permeability. Each gas permeable corrective screen 1 has a thickness of approximately 0.70 millimeters. Each gas permeable corrective screen 1 is made from material, such as polycarbonate material, providing ultraviolet protection. Each gas permeable corrective screen 1 is removable for cleaning.

The hydro-contact lens assembly 100 may include a separate frame 2 to which each gas permeable corrective screen 1 is fixed by way of a rubber gasket 8. The rubber gasket 8 may be dimensioned and adapted to hermetically or fluidly fixing the corrective screen assembly 1 on the frame 2. It also hermetically seals the liquid contents within the liquid chamber formed anteriorly around the eyeball of the human user. The rubber gasket 8 may be made of one hundred percent (100%) silicone material.

Each frame 2 may provide one or more auxiliary inputs/inlets 3. Each auxiliary inputs 3 is a filling port located on the upper outer edge of the frame 2, with respect to each eye socket. The function of these inlets 3 is to enable practitioners to urge the solution or doses of medication (e.g., during ophthalmic medical treatments), thus ensuring that they are not lost at the time of placement.

A closing plug 4 may be provided to close (plug) the auxiliary inlet 3. The closing plugs tapered geometry facilitates a fluid seal forming at the inlet 3. The closing plug 4 may be one hundred percent (100%) silicone material and conform to the plateau.

A suction cup 5 may be connected to the frame 2 proximal the gas permeable corrective screen assembly 1. Both the suction cup 5 and the frame 2 may provide complementary channels 58 and 28, respectively, for facilitating the interface of the suction cup 5 to the frame 2. The channel 58 of the suction cup 5 is provided by an interfacing portion 56.

The suction cup 5 is dimensioned and adapted to adhere to the skin adjacent to the eye sockets and under pressure maintains the seal to define a liquid chamber 10 anterior the eyeball of the human user, wherein liquid is kept inside the liquid chamber 10 without liquid leakage occurring. The use of suction cups 5 avoids exposure to chemical residues resulting from the use of adhesives for adhesion to the skin. The suction cups 5 for the hydro-contact lens assembly 100 will be designed for liquid containment, and the skirts 52 of the suction cups adhere to the facial surface, both internally and externally, very different from the design of diving or swimming masks.

A bridge 6 may join two frames 2 and may be made of one hundred percent (100%) silicone material and must be elastic. It is understood that the hydro-contact lens assembly 100 contemplates the use of only one corrective lens 1 and cooperating frame 2, analogous to an eye patch embodiment.

A strap 7 engages the opposing frames 2 for selectively adjusting around the pressurized head to keep the liquid chamber 10 in the correct position. The strap 7 may be made of one hundred percent (100%) silicone material and must be elastic. The relationship between the corrective shield 1 and its mounting frame 2 contemplates the vertex distance (the distance between the back surface of a corrective lens 1 and the front of the cornea) and the presence of fluid therebetween influencing the dioptric correction. This is directly related to the rubber gaskets, which directly influence the calculation of the angles and distances in front of the eyeball, which allows the support and the liquid content within the liquid chamber 10. These three components are directly related to vision or visual function, and the liquid content to the refraction of light. The relationship of the auxiliary inlets 3 with the frame is to allow the liquid to be poured in such a way that it does not spill, it is recommended that this be done in a vertical position, to achieve the most optimal liquid containment. The relationship of the plug and the auxiliary inlets is to pressurize and depressurize the hydro-contact eyeglass from the atmospheric pressure against the liquid content, releasing the atmospheric pressure of the instrument adhered to the eye at the moment of removing it, even avoiding a liquid drainage or liquid precipitation towards the nasolacrimal duct (explained by the Boyle-Mariotte Law), thus generating more guarantees for the visual health.

Likewise, the stopper 4 prevents the entry of external agents that may contaminate the solution, such as: mites, dust, bacteria, viruses, foreign bodies and natural evaporation, thus avoiding a higher saline concentration (where the liquid content becomes denser). The cap also prevents, in the event of an impact, the generation of atmospheric shock pressure when the goggle is removed. The plug 4 has a diameter and adjustment ratio in relation to the auxiliary inlet 3, since it has an adjustable base that hermetically seals the liquid containment and is easily released by turning or direct insertion. The bridge is interchangeable and is directly related to the frame, since it allows the frame to be anchored.

The bridge 6 collaborates in the adjustment of the shields and suction cups 5, in a more anatomical position of adhesion to the ocular cavity. Likewise, the bridge 6 enables effective and decisive adjustment due to its interchangeability capacity (change of sizes) for making selectively adjustments in view of the pupillary distance; however, these calculations are made previously before assembly, but there is a freedom of adjustment with the bridge 6 that enables such selective adjustment. Also, the bridge 6 has a direct relationship with the suction cups 5 and the frame 2, making the suction cups 5 mold to the facial vault in a strong way, given the fact that they are anchored to the frame 2. The straps 7 are related to the anchor and the frame and are responsible for the compression required to achieve liquid containment through the suction cups 5 and facial vault or cavity defined by the frame 2 and possibly the suction cup 5 that seals against the skin, around the eye sockets.

Facial cavity/liquid chamber 10 bonding connection by way of sealing material (in certain embodiment, the material along the periphery of a skirt 52 of the suction cup 5). Relatedly, the skirts 52 that are projected at an outward and inward angle. The design geometry allows the liquid containment space to be reduced in a more usable way and avoids a displacement zone of liquid leakage.

The present invention embodies a method of fixing the corrective screen component on the frame base and on the fixing rubber seals. The mounting of the corrective screen can be adjusted to the distance to the vertex. It is required to calculate the panoramic angle, pantoscopic angle and facial angle prior to mounting, since it is not advisable to adjust afterwards, as this instrument contains liquid, and any possibility of solution spillage must be avoided.

A liquid chamber 10 is the product of the process of properly wearing the hydro-contact lens assembly 100. The liquid chamber's function is to contain the liquid and it is the corrective medium. This structure is like a screen of an atmospheric isolation telescope.

One of many filling systems are contemplated by the present invention as long as liquid is urged into the liquid cavity, be it gravity-urged through the filler neck or otherwise. It is recommended to tilt the head slightly backwards to avoid air spaces, then each inlet is closed. This process does not generate an additional variant of ocular pressure.

The sealing plug 4 functions to close (plug) the auxiliary inputs 3. The sealing plug 4 is easy to locate by touch and canalizes the solution filling due to its geometry.

Distance to the corrective screen vertex: the distance to the vertex will generally be as close to the eye as possible (without having physical contact with the eyelids) can be adjusted by tightening and loosening of the strap 7.

A method of making the present invention may include the following. A manufacture would understand that the hydro-contact lens assembly 100 is not for generic use, and so must be custom made for each person depending on their visual condition, and only a technician specialized in optometry and contact lens, with the given specifications of the invention, optical health management, training and optometric calculations, could adapt it.

The material used for the permeable corrective screen assembly 1 is a critical element, but it can also be improved by increasing the permeability with the use of another material that will improve the existing oxygen transmissibility.

The only way to be able to shuffle, exchange or reconfigure some element or component of the invention, would be to improve the initial design of the prototype, that goes more in line with the anatomy, as well as at the manufacturing level, to be able to have access to materials of permeable characteristics of larger diameters for the corrective screen 1 that allows a larger field of view. The present invention contemplates the structure or frame 2 to be attached to the facial vault or eyelids without using the components of the suction cup, bridge, and strap as they are thought in the initial prototype.

A method of suing the present invention may include the following. First, the user may disassemble the corrective shields for cleaning and wash them thoroughly with products recommended for the contact lens industry for gas permeable materials. Second, the user must wash the structure or frame very well, preferably with water and hypoallergenic soap. Third, the user rinses with plenty of water, both the corrective screens and the frame (steps one and two). Fourth, the user may remove the sealing plugs. Fifth, the user may install the corrective screens on the structure. Sixth, the user may place the assembled structure around the head in the primary gaze position. It is very important that the user removes the closing caps beforehand. Seventh, the user may adjust the suction cups around the eye sockets, fastening correctly by means of an elastic strap to the head, with sufficient pressure so that there is no leakage during liquid filling. Eighth, the user may pour the isotonic solution from the upper part through the auxiliary inlets, once the liquid chamber 10 is full, it is recommended to wait a few minutes (subject to clinical studies) for further thermal compensation of the solution and close the filling inlets with the closing plugs. Ninth, once the device is in place, the user may verify that it is filled with the solution and that it is securely fastened. If necessary, the user may have to tilt the head slightly backwards or sideways to level the filling of the solution. Tenth, the times of use will be at the discretion of the physician by subsequent clinical studies, which will determine the most appropriate recommendations. Eleventh, before removing the hydro-contact lens assembly 100's seal material/suction cup 5, each auxiliary inlet must be opened to release the pressure before removal.

Additionally, the present invention was devised for the field of visual health, originally aimed at correcting atypical visual defects, as well as affections of the visual organ. It is feasible that its elements or parts can be fabricated through the direction of a computer with the appropriate technology. However, the realization of each device is customized for everyone, and at no time can it be of standardized manufacture.

As used in this application, the term “about” or “approximately” refers to a range of values within plus or minus 10% of the specified number. And the term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel, substantially parallel, or forming an angle of less than 35.0 degrees. For purposes of this disclosure, the term “transverse” means perpendicular, substantially perpendicular, or forming an angle between 55.0 and 125.0 degrees. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down,” and the like, are words of convenience and are not to be construed as limiting terms unless specifically stated to the contrary.

It should be understood, of course, that the foregoing relates to exemplary embodiments of the invention and that modifications may be made without departing from the spirit and scope of the invention as set forth in the following claims.

What is claimed is:

1. An eye treatment device comprising:
a goggle frame, having at least one cavity, configured to surround and be spaced from an eye;
a corrective lens along a face of the goggle frame; and
an inlet formed in an upper portion of the goggle frame, the inlet fluidly communicating the cavity to an external environment.

2. The eye treatment device of claim 1, further comprising:
a sealing material along a perimeter of the goggle frame for forming a hermetical seal against skin around a socket of the eye.

3. The eye treatment device of claim 2, wherein the sealing material is a portion of a suction cup.

4. The eye treatment device of claim 3, further comprising:
a tapered stopper for fluidly sealing the inlet.

5. The eye treatment device of claim 4, wherein the corrective lens is made from gas permeable material having a thickness of approximately 0.70 millimeters.

6. The eye treatment device of claim 5, wherein a distance between a distance between a back surface of the corrective lens and a front of a of the eye is selected as function of a radius of curvature of the corrective lens.

7. A method of treating dry eye syndrome, the method comprising:
providing the eye treatment device of claims 1; and filling the cavity with a fluid by way of the inlet.
